(19) United States
(12) Patent Application Publication
(10) Pub. No.: US 2014/0277034 A1
(43) Pub. Date: Sep. 18, 2014

(54) SHEATH COUPLING MEMBER AND ASSOCIATED INSTRUMENT ASSEMBLY

(71) Applicant: Misonix, Incorporated, Farmingdale, NY (US)

(72) Inventor: ALEXANDER L. DARIAN, Brightwaters, NY (US)

(73) Assignee: Misonix, Incorporated, Farmingdale, NY (US)

(21) Appl. No.: 14/211,656

(22) Filed: Mar. 14, 2014

Related U.S. Application Data
(60) Provisional application No. 61/792,893, filed on Mar. 15, 2013.

(51) Int. Cl. A61B 17/32

(52) U.S. Cl. CPC A61B 17/320068
USPC 606/169

(57) ABSTRACT

A coupling member for an ultrasonic instrument assembly includes a body member having a first port at a first end and a second port at a second end opposite the first end, the body member having a lumen or channel extending from the first end to the second end so that the first and the second port communicate with one another. The body member is made of a flexible resilient material and is provided with at least one protrusion (or projection) extending into the lumen or channel. The protrusion is variously insertable into at least one cooperating groove or recess on an outer surface of a tubular member to enable an adjustment in a degree of insertion of the tubular member into the coupling member.

### BACKGROUND OF THE INVENTION

This invention pertains to surgical instruments, particularly to ultrasonic instrument assemblies and more particularly to such instrument assemblies with a sheath.

Ultrasonic medical instruments typically comprise a handpiece, a probe, and a tubular sheath around the probe. The handpiece contains a magnetostrictive or piezoceramic transducer that converts an incoming electrical alternating waveform of ultrasonic frequency into a mechanical vibration that is conducted through the probe, generating a standing wave therein and causing an operative tip or end effector surface of the probe, located at a vibration anti-node, to vibrate at the ultrasonic frequency. The ultrasonic probe is frequently formed with a longitudinal lumen that communicates via a conduit in the ultrasonic transducer with a source of saline solution, optionally containing one or more chemicals for anesthesia, sterilization, etc. The principal function of the liquid solution is cooling the ultrasonic probe and the tissues with which it is in contact. In addition, the liquid may function as a carrier or transport medium for organic debris that is produced upon the application of ultrasonic mechanical energy to a surgical site. The liquid with the debris is typically removed from the surgical site by means of a suction or vacuum generator connected to an annular lumen between the probe and the sheath. Alternatively, the suction source may be connected to the probe lumen and the liquid source to the space between the probe and the sheath.

It is not uncommon for the instrument assembly to be assembled and reassembled in the operating room. Sometimes different probes are successively connected to the same handpiece. If there are difficulties in properly forming the attachments among the different pieces of the instrument assembly, an inefficient operation may result, either compromising the effectiveness of the surgery or extending the operating time.

### OBJECTS OF THE INVENTION

It is an object of the present invention to provide for an improved assembly of handpiece, probe and sheath to one another. More particularly, the invention contemplates a connector component for facilitating an acceptable formation of the sheath.

It is a more specific object of the present invention to provide a watertight attachment between distal and proximal sheath parts, and to provide spacing/length adjustment between them.

Another specific object of the present invention is to provide, for an ultrasonic instrument assembly, a coupling member or sheath connector that facilitates adaptation of the instrument assembly to the particular user contingencies. It is contemplated that such a coupling member or sheath connector enable a variation in the position of the distal end of a distal sheath portion relative to the operative tip of the probe.

### SUMMARY OF THE INVENTION

An ultrasonic instrument assembly in accordance with the present invention comprises a handpiece, a probe or instrument shaft extending from one end of the handpiece, a proximal sheath portion, a distal sheath portion, and a tubular coupling member or sheath connector. The proximal sheath portion is connected to the handpiece and surrounds a proximal end portion of the probe or instrument shaft. The distal sheath portion surrounds a distal end portion of the probe or instrument shaft. The tubular coupling member has a lumen or channel, a proximal end section of the coupling member being disposed over and in substantially fluid tight contact with the proximal sheath portion, and a distal end section of the coupling member being disposed over and in substantially fluid tight contact with the distal sheath portion. At least one of the proximal sheath portion and the distal sheath portion is provided on an outer surface with at least one recess or groove which mates with at least one projection extending from the body of the coupling member or sheath connector into the lumen or channel. The projection is variously insertable into the recess or groove of the relevant sheath portion to enable or accommodate an adjustment in the spacing between the proximal sheath portion and the distal sheath portion.

In a particular embodiment of the invention, the projection is one of a plurality of mutually spaced projections on a main body of the coupling member or sheath connector. The projections all extend into the lumen or channel of the coupling member and are spaced from one another at least in a longitudinal or axial direction between the first port and the second port. The spacing of the projections allows for alternate insertion of the projections into the recess or groove on the outer surface of the one sheath portion, thereby changing the effective length of the coupling member to match different spacings between the distal end of the proximal sheath portion and the proximal end of the distal sheath portion. Thus, the coupling member accommodates multiple discrete inter-sheath distances or gaps. The differences in the gap sizes are equal to the longitudinal or axial distances between longitudinally or axially adjacent ones of the projections.

Pursuant to a more particular embodiment of the invention, the projections are spaced from one another in both the longitudinal or axial direction and in a circumferential direction, thereby forming a cylindrical array of spaced projections. In this embodiment the different inter-sheath distances correspond to respective circular arrays of the locking projections.

Preferably the projections are in the form of nubs or bumps. However, other interior protrusion geometries are possible, such as circumferentially elongated, truncated and increased in number, spirally arranged to allow for a screw-type action, or fully annular rings.

The coupling member may be made of silicone rubber.

The purpose or function of the coupling member or sheath connector is twofold: to provide a watertight attachment between the distal and proximal housings, and to provide spacing/length adjustment between them. The coupling member or sheath connector thus facilitates adaptation of the instrument assembly to the particular user contingencies, for example by enabling a variation in the position of the distal end of the distal sheath portion relative to the operative tip of the probe.

The coupling member or sheath connector closes or completes a cylindrical irrigation and/or suction channel bounded on the inside by the probe and on the outside by the sheath elements. The seal on the distal side of the coupling member or sheath connector is achieved mainly by the diameter of the main ID at the base of those protrusions of the silicone connector, with respect to the OD of the distal mating part. The proximal seal is achieved in the same way (just no protrusions).

A coupling member for an ultrasonic instrument assembly comprises, in accordance with the present invention, a body member having a first port at a first end and a second port at a second end opposite the first end, the body member having a lumen or channel extending from the first end to the second end so that the first and the second port communicate with one another. The body member is made of a flexible resilient material and is provided with at least one protrusion (or projection) extending into the lumen or channel. The protrusion is variously insertable into at least one cooperating groove or recess on an outer surface of a tubular member to enable an adjustment in a degree of insertion of the tubular member into the coupling member.

The protrusion may be one of a plurality of mutually spaced projections on the main body extending into the lumen or channel, where the projections are spaced from one another at least in a longitudinal or axial direction between the first port and the second port. In addition, the projections may spaced from one another in a circumferential direction to form a cylindrical array of spaced projections.

### BRIEF DESCRIPTION OF THE DRAWING

FIG. 1 is a side elevational view of an ultrasonic instrument assembly in accordance with the present invention.

FIG. 2 is a partial side elevational view, on an enlarged scale, of the ultrasonic instrument assembly of FIG. 1, showing a coupling member or sheath connector in accordance with the present invention.

FIG. 3 is an end elevational view of the coupling member or sheath connector of FIGS. 1 and 2.

FIG. 4 is a longitudinal cross-sectional view taken along line IV-IV in FIG. 3

FIG. 5 is a cut-away perspective or isometric view of the coupling member or sheath connector of FIGS. 1-4.

### DETAILED DESCRIPTION

An ultrasonic instrument assembly 10 comprises a handpiece 12, a probe or instrument shaft 18 extending from one end of the handpiece, a proximal sheath portion 14, a distal sheath portion 16, and a tubular silicone coupling member or sheath connector 20. Proximal sheath portion 14 is connected to handpiece 12 and surrounds a proximal end portion of probe or instrument shaft 18. Distal sheath portion 16 surrounds a distal end portion of the probe or instrument shaft. Coupling member 20 has a lumen or channel 22, a proximal end section (not separately designated) of the coupling member being disposed over and in substantially fluid tight contact with proximal sheath portion 14, and a distal end section (not separately designated) of the coupling member being disposed over and in substantially fluid tight contact with distal sheath portion 16. At least one of the sheath portions 14 and 16, for instance, distal sheath portion 16, is provided on an outer surface with at least one recess or groove 24 which mates with at least one and preferably a multiplicity of protrusions or projections 26 extending from the body (not separately designated) of coupling member 20 into the lumen or channel 22 thereof. Protrusions or projections 26 are variously insertable into recess or groove 24 of sheath portion 16 to enable or accommodate an adjustment in the spacing between the proximal sheath portion 14 and the distal sheath portion 16.

Protrusions or projections 26 are mutually spaced in both a longitudinal or axial direction and a circumferential direction to form a cylindrical array of locking projections. The spacing of the projections 26 allows for alternate insertion of the projections into recess or groove 24 on the outer surface of distal sheath portion 16, thereby changing the effective length of the coupling member 20 to match different spacings between the distal end of the proximal sheath portion 14 and the proximal end of the distal sheath portion 16. The coupling member 20 thereby accommodates plural discrete inter-sheath distances or gaps. The differences in the gap sizes are equal to the longitudinal or axial distances between longitudinally or axially adjacent ones of the projections 26.

Protrusions or projections 26 are in the form of nubs or bumps. However, other interior protrusion geometries are possible, such as circumferentially elongated, truncated and increased in number, spirally arranged to allow for a screw-type action, or fully annular rings.

The purpose or function of coupling member or sheath connector 20 is twofold: to provide a watertight attachment between the distal and proximal sheath portions 14 and 16, and to provide spacing/length adjustment between them. Coupling member or sheath connector 20 thus facilitates adaptation of the instrument assembly to the particular user contingencies, for example by enabling a variation in the position of the distal end of the distal sheath portion 16 relative to the operative tip of the probe 18 at 28. This allows for variation in the degree of extension of the probe's operative tip from the sheath portion 16 and may allow for complete coverage of the probe tip, when the tip is located inside the distal sheath portion 16.

Coupling member or sheath connector 20 closes or completes a cylindrical irrigation and/or suction channel bounded on the inside by probe 18 and on the outside by the sheath portions 14 and 16. The seal on the distal side of coupling member or sheath connector 20 is achieved mainly by the diameter of the main ID at the base of those protrusions of the silicone connector, with respect to the OD of the distal mating part. The proximal seal is achieved in the same way (just no protrusions).

Coupling member comprises a body member (not separately designated) having a first port 30 at a first end and a second port 32 at a second end opposite the first end. Lumen or channel 22 extends from first end 30 to second end 32 so that the ports communicate with one another.

Coupling member 20 has a bulbous shape, with a waist section 34 that is wider than either end. The outer surface exhibits a gentle convex curve tapering at the opposite ends of the coupling member.

Although the invention has been described in terms of particular embodiments and applications, one of ordinary skill in the art, in light of this teaching, can generate additional embodiments and modifications without departing from the spirit of or exceeding the scope of the claimed invention. For example, the projections 26 may be located on the outer surface of one or both sheath portions 14 and 16, while the cooperating recess or groove 24 is located in lumen or channel 22 of coupling member 20. Accordingly, it is to be understood that the drawings and descriptions herein are proffered by way of example to facilitate comprehension of the invention and should not be construed to limit the scope thereof.

What is claimed is:

1. A coupling member for an ultrasonic instrument assembly, comprising a body member having a first port at a first end and a second port at a second end opposite said first end, said body member having a lumen or channel extending from said first end to said second end so that said first and said second port communicate with one another, said body member being made of a flexible resilient material, said body member being provided with at least one protrusion extending into said lumen or channel, said at least one protrusion being variously insertable into at least one cooperating groove or recess on an outer surface of a tubular member to enable an adjustment in a degree of insertion of said tubular member into said coupling member.

2. The coupling member set forth in claim 1 wherein said at least one protrusion is one of a plurality of mutually spaced projections on said main body extending into said lumen or channel, said projections being spaced from one another at least in a longitudinal or axial direction between said first port and said second port.

3. The coupling member set forth in claim 2 wherein said projections are spaced from one another in said longitudinal or axial direction and in a circumferential direction to form a cylindrical array of spaced projections.

4. The coupling member set forth in claim 3 wherein said projections are in the form of nubs or bumps.

5. The coupling member set forth in claim 1 wherein said body member is made of silicone rubber.

6. An ultrasonic instrument assembly comprising: a handpiece; a probe or instrument shaft extending from one end of said handpiece; a proximal sheath portion connected to said handpiece and surrounding a proximal end portion of said probe or instrument shaft; a distal sheath portion surrounding a distal end portion of said probe or instrument shaft; and a tubular coupling member with a lumen or channel, a proximal end section of said coupling member being disposed over and in substantially fluid tight contact with said proximal sheath portion, a distal end section of said coupling member being disposed over and in substantially fluid tight contact with said distal sheath portion, at least one of said proximal sheath portion and said distal sheath portion being provided on an outer surface with at least one recess or groove, said coupling member having at least one projection extending into said lumen or channel and being variously insertable into said at least one recess or groove to enable or accommodate an adjustment in a spacing between said proximal sheath portion and said distal sheath portion.

7. The ultrasonic instrument assembly set forth in claim 6 wherein said lumen or channel extends between a first port at one end of the coupling member and a second port at another end of the coupling member, said coupling member having a main body, said at least one projection being one of a plurality of mutually spaced projections on said main body extending into said lumen or channel, said projections being spaced from one another at least in a longitudinal or axial direction between said first port and said second port.

8. The ultrasonic instrument assembly set forth in claim 7 wherein said projections are spaced from one another in said longitudinal or axial direction and in a circumferential direction to form a cylindrical array of spaced projections.

9. The ultrasonic instrument assembly set forth in claim 8 wherein said projections are in the form of nubs or bumps.

10. The ultrasonic instrument assembly set forth in claim 6 wherein said body member is made of silicone rubber.
